(19) United States
(12) Patent Application Publication
(10) Pub. No.: US 2021/0220009 A1
(43) Pub. Date: Jul. 22, 2021

(54) Surgical retractor fixation device/shim

(71) Applicant: BHDL Holdings, LLC, Atlanta, GA (US)

(72) Inventors: Bradley A. Heiges, Savannah, GA (US); David E. Lane, II, Atlanta, GA (US)

(21) Appl. No.: 17/104,629

(22) Filed: Nov. 25, 2020

Related U.S. Application Data
(60) Provisional application No. 62/939,804, filed on Nov. 25, 2019.
(60) Provisional application No. 62/940,677, filed on Nov. 26, 2019.

(51) Int. Cl. A61B 17/34; A61B 90/35; A61B 17/02

(52) U.S. Cl. CPC A61B 17/34; A61B 90/35; A61B 2090/309; A61B 17/0218; A61B 17/025

(57) ABSTRACT

A two-piece surgical retractor fixation device/shim for use in surgical procedures, particularly spinal surgeries. The surgical retractor fixation device/shim is designed to lock a retractor to an anatomical structure, such as the track of the vertebral body.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 62/939,804 entitled “SURGICAL RETRACTOR FIXATION DEVICE/SHIM” filed Nov. 25, 2019 and to U.S. Provisional patent Application No. 62/940,677 entitled “SURGICAL RETRACTOR FIXATION DEVICE/SHIM” filed Nov. 26, 2019. The contents of the above referenced applications are incorporated herein by reference in its entirety.

### FIELD OF THE INVENTION

The present system and method relate to devices and methods for performing percutaneous surgeries, and in particular, to fixation devices for use in various surgical procedures, such as spinal surgeries.

### BACKGROUND OF THE INVENTION

Open spinal surgical procedures generally require a relatively long incision, extensive muscle stripping, prolonged retraction of tissues, and increased risk of damage to vascular and nerve tissue. This type of procedure usually necessitates many weeks of post-operative recovery due to long hours under general anesthesia, blood transfusions and the unavoidable trauma caused to the body tissues during the procedure. An open surgical procedure will also result in significant permanent scarring, leading to fusion disease.

Surgery performed percutaneously has achieved major improvements over open surgery. The reduction of muscle and tissue dissection significantly reduces post operative recovery, pain, and recovery time. Percutaneous surgery is particularly beneficial for spinal surgery because the surgical area is deep within the body and in locations surrounded by sensitive and critical body tissues. Tube retractors have been developed to provide minimally invasive access to the surgical area. The ability to dilate muscle tissue, as opposed to strip or detach them from the bony anatomy, will reduce the damage and risks normally associated with the open type surgery.

The typical tube retractor technique starts with the identification of the correct entry point, establishing the trajectory from the skin to the pathology to be addressed and the corresponding skin incision. The initial soft tissue dilator is inserted through the incision and forcefully advanced to the objective site. A series of larger dilators are inserted over the initial dilator, thereby sequentially increasing the diameter until the final/operative dilator is inserted. Once the operative dilator is in place, it must be fixed in order to resist movement that will result from forces imposed by the patient's tissue. Currently, the accepted approach to fixation is a point outside of the patient's anatomy. Typically, a rigid arm is attached at one end to the retractor, while the opposite end of the arm is attached to a bed rail clamp. Once the retractor is fixed in position, the surgeon begins the operation to address the pathology. Upon completion of the procedure, the retractor tube is removed and the skin incision is closed. Because of the reduced morbidity to the patient, the patient's initial recovery time, blood loss, operating room time, anesthesia time, stay in the hospital, recovery time, and the overall cost of the procedure should be less.

One of the most difficult aspects of the current technique is that the rigid fixation of the retractor is sometimes subject to unintentional or unavoidable movement of the patient during the course of the surgical procedure. To fix the retractor and prevent movement, typical surgical procedures require the use of screws to fix the retractor in place. Use of screws is not ideal, as they increase the risk of bleeding outside the disc space and introduce sharp edges to the anatomical structures. Use of screws to fix the retractor in place is also problematic in that they are tedious to insert and remove.

### SUMMARY OF THE INVENTION

The present invention is directed to methods and devices for performing percutaneous, minimally invasive spinal surgery. In particular, the invention includes an improved surgical retractor fixation device/shim, and methods of use, for use with a retractor device. The surgical retractor fixation device/shim is designed to lock any retractor to an anatomical structure, such as via the track of the intradiscal space between vertebral bodies. The surgical retractor fixation device/shim includes an outer member and an inner member. The inner member comprises one or more anatomical structure engaging members, such as a pair of unidirectional opposing flanges or cleats that can be deployed out through openings within the outer member to engage with, or bite into, the vertebral body endplate. The unidirectional opposing flanges or cleats can be designed to fold back, inwards or in line with the surgical retractor fixation device/shim, thus unlocking the flanges or cleats and allowing the surgical retractor fixation device/shim to be removed, which detaches the retractor from the lateral aspect of the vertebral body.

The surgical retractor fixation device/shim addresses the current problem in which retractors, when used for certain surgical procedures, are fixed to the vertebral body using bone screws. Use of screws in this capacity increases the risk of bleeding, particularly outside the disc space, and introduces a sharp edge to the disc space. To avoid these issues, the surgical retractor fixation device/shim according to the invention is designed to be used internal to the disc space, thus reducing the need for using screws in the bone. The surgical retractor fixation device/shim acts as an internal anchor that prevents the retractor from being pulled out or away.

Accordingly, it is an objective of the invention to provide a surgical retractor fixation device/shim for use with a retractor.

It is a further objective of the invention to provide a surgical retractor fixation device/shim that reduces the risk of internal bleeding.

It is a further objective of the invention to provide a surgical retractor fixation device/shim that reduces the use of sharp edges within the disc space.

It is yet another objective of the invention to provide a surgical retractor fixation device/shim that locks to the vertebral body inside the disc space.

It is a still further objective of the invention to provide a surgical retractor fixation device/shim that aligns a blade to the intra-vertebral disc space.

It is a still further objective of the invention to provide a surgical retractor fixation device/shim that locks to the disc space, preventing left/right movement.

It is a still further objective of the invention to provide a surgical retractor fixation device/shim that locks to the disc space, preventing the retractor from pulling out or away.

It is a still further objective of the invention to provide a surgical retractor fixation device/shim that locks to the disc space, preventing left/right movement and preventing the retractor from pulling out or away.

Other objects and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a perspective view of an illustrative embodiment of a surgical retractor fixation device/shim;

FIG. 2 is an alternative perspective view of the surgical retractor fixation device/shim;

FIG. 3 is a left view of the surgical retractor fixation device/shim;

FIG. 4 is a right view of the surgical retractor fixation device/shim;

FIG. 5 is a front view of the surgical retractor fixation device/shim;

FIG. 6 is a perspective view of an illustrative embodiment of an inner member of the surgical retractor fixation device/shim, shown in a first, or non-deployed position;

FIG. 7A is a perspective view of the inner member of the surgical retractor fixation device/shim, shown in a second, or deployed position;

FIG. 7B illustrates an embodiment of the inner member of the surgical retractor fixation device/shim shown with four anatomical structure engaging members;

FIG. 8 illustrates the surgical retractor fixation device/shim, shown with the inner member removed from the outer member;

FIG. 9 illustrates the surgical retractor fixation device/shim, shown with the inner member inserted and resting within the interior of the outer member;

FIG. 10A illustrates the surgical retractor fixation device/shim shown in a first use position prior to deployment;

FIG. 10B illustrates the surgical retractor fixation device/shim shown in a second use positon;

FIG. 10C illustrates the surgical retractor fixation device/shim shown in a third use positon, with the inner member in a deployed position;

FIG. 11A illustrates an alternative embodiment of the surgical retractor fixation device/shim, shown with inner member guides;

FIG. 11B illustrates the inner member shown in FIG. 11A, shown in a deployed state;

FIG. 12A illustrates the surgical retractor fixation device/shim used in a top down method, shown in a first position;

FIG. 12B illustrates the surgical retractor fixation device/shim used in a top down method, shown in a second position prior to deployment of the inner member;

FIG. 12C illustrates the surgical retractor fixation device/shim used in a top down method, shown in a third position, during deployment of the inner member;

FIG. 13 illustrates the surgical retractor fixation device/shim inserted into the disc;

FIG. 14 illustrates the surgical retractor fixation device/shim inserted into the disc space, with the inner member deployed and engaging with adjacent vertebral bodies;

FIG. 15 is a perspective view of an illustrative embodiment of an adjustable retractor;

FIG. 16 is a perspective view of the adjustable retractor illustrated in FIG. 15, shown with the second pair of elongated members removed;

FIG. 17 is a perspective view of an elongated member of a first pair of elongated members;

FIG. 18 is a perspective view of a second pair of elongated members;

FIG. 19 is an alternative view of the second pair of elongated members shown in FIG. 18;

FIG. 20 is a top view of the adjustable retractor illustrated in FIG. 15;

FIG. 21 is a perspective view of the adjustable retractor engaged with a hand tool;

FIG. 22 is a perspective view of the hand tool;

FIG. 23 is an alternative embodiment of the adjustable retractor illustrated in FIG. 15;

FIG. 24 is a perspective view of a series of initial dilators for use with the adjustable retractor illustrated in FIG. 15 or FIG. 23;

FIG. 25 is a top perspective view of an illustrative embodiment of a retractor shaped sequential dilator inserted into the adjustable retractor illustrated in FIG. 15;

FIG. 26 is a bottom perspective view of the illustrative embodiment of a retractor shaped sequential dilator inserted into the adjustable retractor illustrated in FIG. 15;

FIG. 27 is a perspective top view of the retractor, a patient pad, and a device to anchor the retractor to the patient pad;

FIG. 28 is a top perspective view of an alternative arrangement for anchoring the retractor to the patient pad;

FIG. 29 is a side perspective view of another alternative embodiment to anchor the retractor to the patient pad;

FIG. 30 is a side perspective view of yet another alternative embodiment to anchor the retractor to the patient pad;

FIG. 31 is a side perspective view of a retractor including a handle to manually stabilize the retractor in the patient;

FIG. 32 illustrates a system for providing access to a surgical location within a patient, the system including a retractor and the surgical retractor fixation device/shim;

FIG. 33 illustrates the system for providing access to a surgical location within a patient in which the surgical retractor fixation device/shim is inserted into and engages with a retractor body

FIG. 34 illustrates the surgical retractor fixation device/shim with a cannulated passageway;

FIG. 35 is a cross-sectional view of the surgical retractor fixation device/shim with cannulated passageway shown in FIG. 34;

FIG. 36 illustrates the surgical retractor fixation device/shim with cannulated passageway, shown with the cannulated passageway in line with the outer member main body surface openings;

FIG. 37 is a cross-sectional view of the surgical retractor fixation device/shim with cannulated passageway shown in FIG. 36;

FIG. 38 illustrates the surgical retractor fixation device/shim shown in FIG. 34 with the surgical retractor fixation device/shim inner member 14 inserted;

FIG. 39 illustrates the surgical retractor fixation device/shim with cannulated passageway having a light source inserted therein;

FIG. 40 illustrates the surgical retractor fixation device/shim with cannulated passageway having a suction or vacuum tube inserted therein; and

FIG. 41 illustrates the surgical retractor fixation device/shim with cannulated passageway having a camera or camera lens inserted therein.

### DETAILED DESCRIPTION OF THE INVENTION

The present invention is directed to methods and devices for performing percutaneous surgery, particularly spinal surgery. The device includes a two-component surgical retractor fixation device/shim designed to lock a retractor to an anatomical structure, such as a vertebral body, or two vertebral bodies simultaneously. The shim includes an outer member and an inner member.

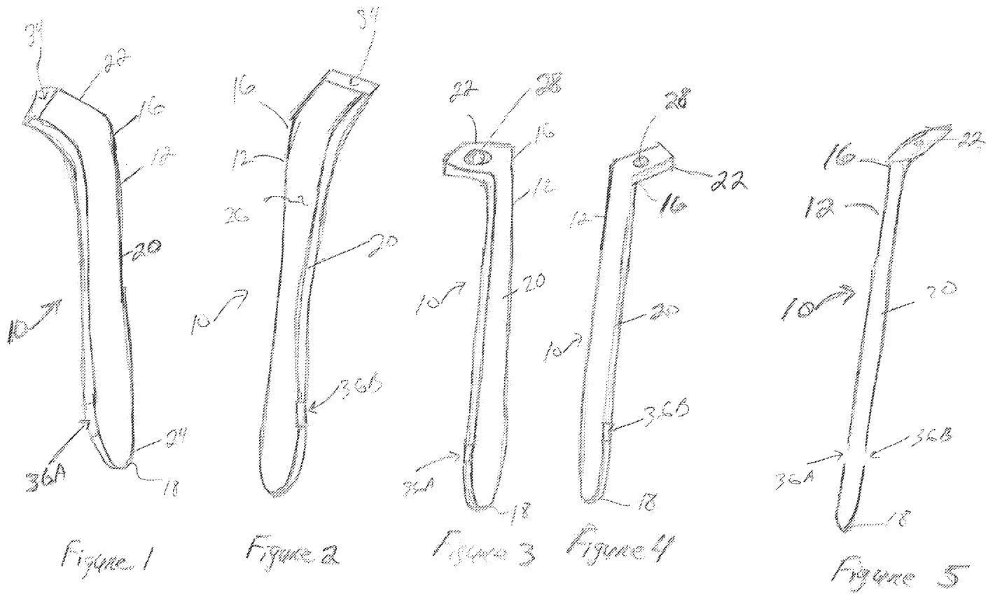

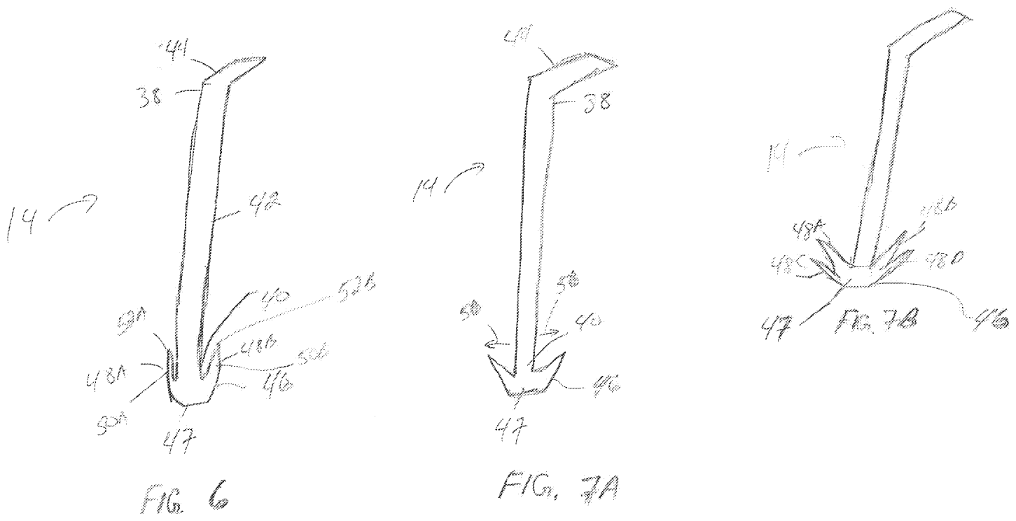

Referring to FIGS. 1-5, an illustrative embodiment of a surgical retractor fixation device/shim 10. The surgical retractor fixation device/shim 10 is designed to provide stability when used with any retractor surgical instrument and provide fixation at multiple points, eliminating or reducing the risk of retractor left/right movement and/or the retractor pulling out or away. While the surgical retractor fixation device/shim 10 is described herein for use with or in spinal surgeries or procedures, such use is for illustrative purpose only. The surgical retractor fixation device/shim 10 may be used in any medical procedure as needed. The surgical retractor fixation device/shim 10 includes an outer member 12 and an inner member 14, see FIG. 8 and FIG. 9. The surgical retractor fixation device/shim 10 is designed to secure to one or more anatomical structures, allowing a portion of the surgical retractor fixation device/shim 10 to act as an internal anchor by securing internally to, for example, the disc space of the spine. Once inserted and deployed or activated, the surgical retractor fixation device/shim 10 prevents the retractor from being pulled out or away from the vertebral body. All components of the surgical retractor fixation device/shim 10 may be made of one or combinations of materials known to one of skill in the art that allows the surgical retractor fixation device/shim 10 to perform its intended function in its intended environment.

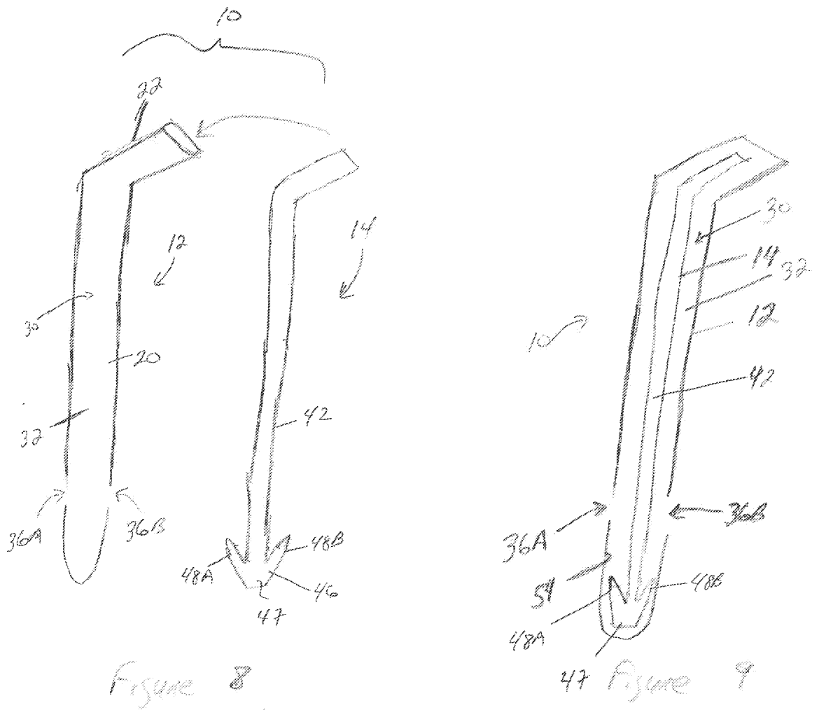

The surgical retractor fixation device/shim 10 is configured to engage and fit or secure within at least a portion of any retractor. The fixation device/shim 10 outer member 12 comprises a first or proximal end 16, a second opposing or distal end 18, and an outer member main body 20 there between. The first end 16 may comprise a handle portion 22 sized and shaped to interact with a portion of a retractor. The second end 18 may comprise a tapered portion 24 and is configured to engage with a portion of a disc, such as the disc annulus. The outer member main body 20 comprises a length that is sufficient to extend the length of, and out past the distal end of a retractor. Accordingly, the length of the surgical retractor fixation device/shim 10 is preferably greater than the length of a retractor which it engages. The length of the surgical retractor fixation device/shim 10 can be designed to be sufficient, such that, when used with a retractor, the second end 18 can extend inside the lateral aspect of the annulus of the disc and extend into the disc itself. Depending on the need of use, the length can vary to allow for insertion just inside the annulus to a length that can cross to the contra-lateral annulus, or anywhere in between. The outer member main body 20 further comprises an outer surface 26. The surgical retractor fixation device/shim 10 may also be configured to be mountable to a table. In such an embodiment, the first end 16 may contain a threaded opening 28, see FIGS. 3 and 4.

The outer member main body 20 comprises an internal lumen 30, see FIG. 9. The internal lumen 30 is hollow or partially hollow to define an interior region 32. The interior region 32 is sized and shaped to receive and allow the surgical retractor fixation device/shim inner member 14 to fit and move within. As such, the first end 16 may comprise an opening 34 leading to the internal lumen 30 and interior region 32. The opening 34 is sized to allow the surgical retractor fixation device/shim inner member 14 to be inserted into the surgical retractor fixation device/shim outer member 12 and allow for a user, such as a surgeon, to access or grab a portion of the surgical retractor fixation device/shim inner member 14 and cause it to move in a linear up/down movement within the interior region 32. Positioned within the outer member main body 20 are outer member main body surface openings 36A and 36B. Both the outer member main body surface opening 36A and the outer member main body surface opening 36B are sized and shaped to allow a portion of the surgical retractor fixation device/shim inner member 14 to extend through and rest within. While the outer member main body surface openings 36A and 36B are positioned towards the second end 18, they may be placed anywhere within the outer member main body. In an illustrative embodiment, the outer member main body surface openings 36A and 36B may be spaced one hundred eighty (180) degrees apart. In addition, the outer member main body surface openings 36A and 36B may assume any shape, including elongated or having a slot configuration. While the outer member main body 20 is illustrated with two outer member main body surface openings, 36A and 36B, three openings, four openings, or more may be used. Alternatively, the outer member main body 20 may comprise a single outer member main body surface opening 36A or 36B.

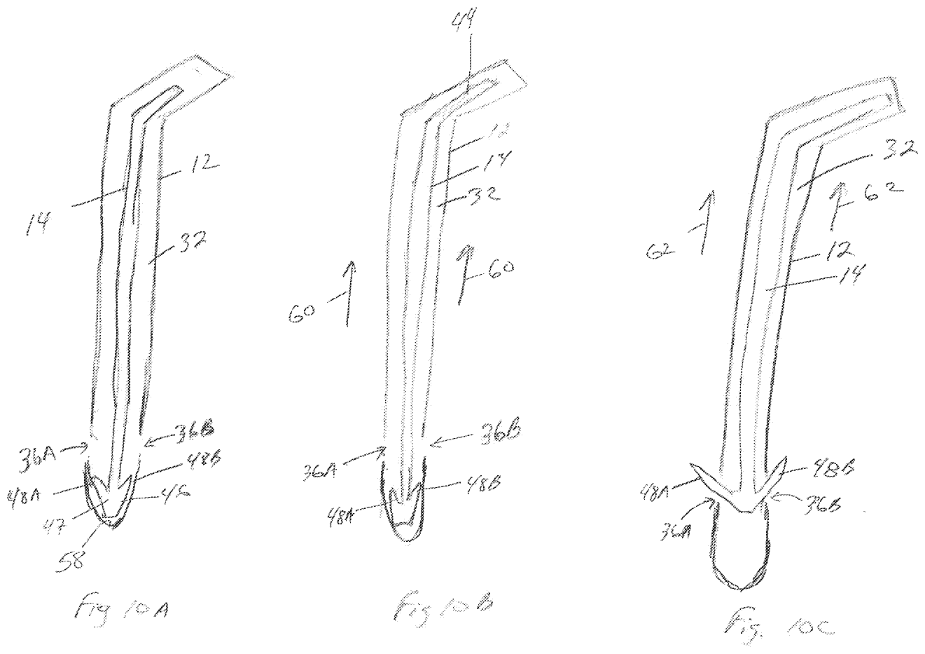

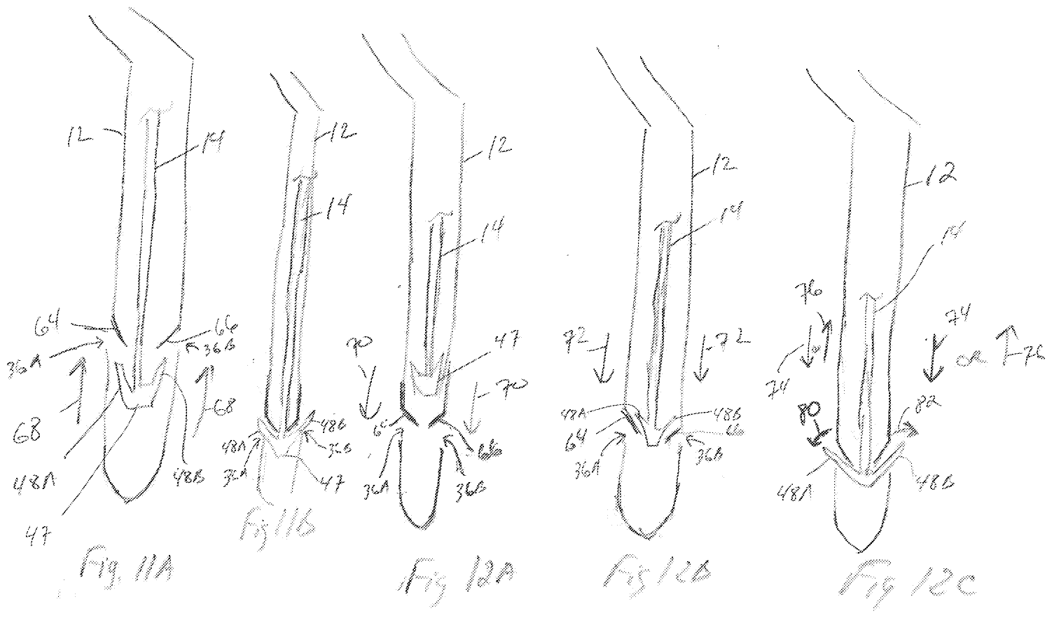

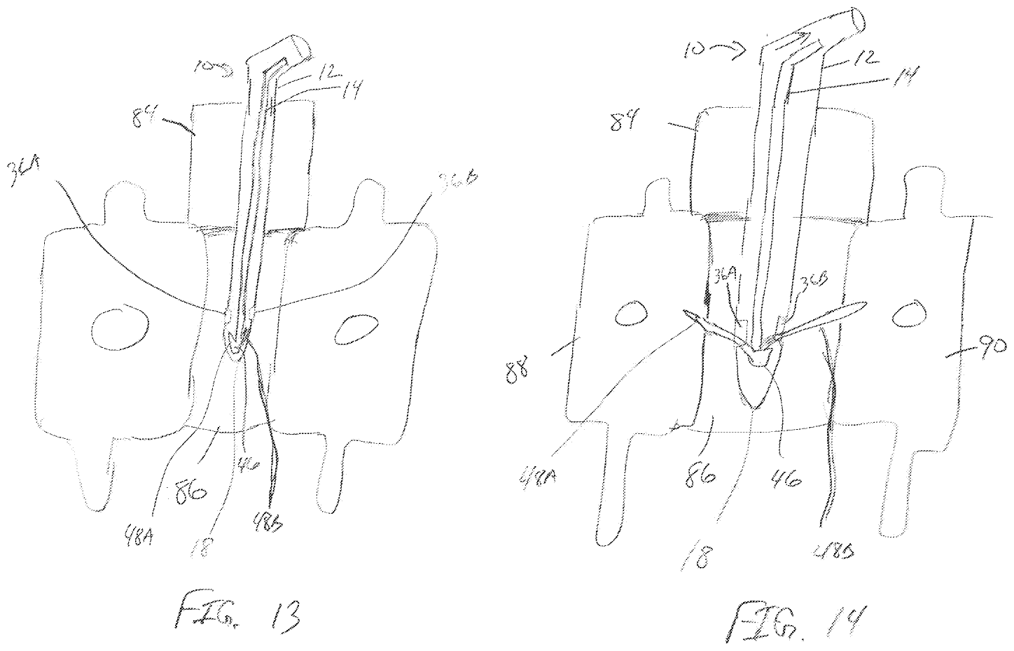

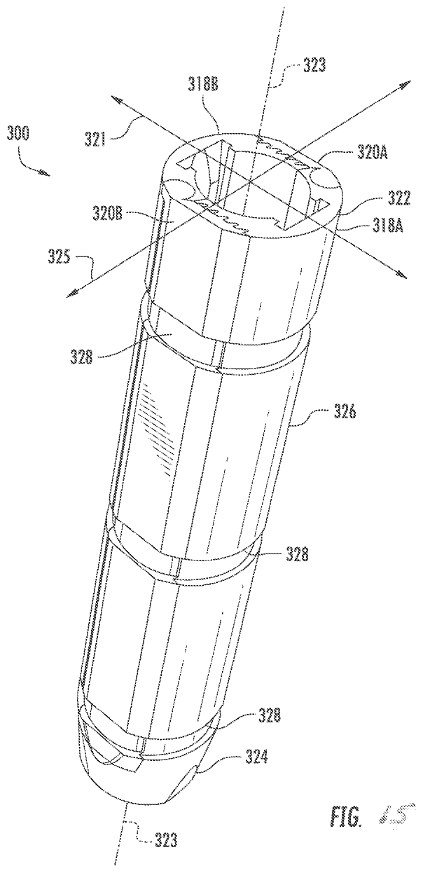

Referring to FIGS. 6 and 7A, an illustrative embodiment of the surgical retractor fixation device/shim inner member 14 is shown. The surgical retractor fixation device/shim inner member 14 is configured to engage and fit or secure within the surgical retractor fixation device/shim outer member 12. The fixation device/shim inner member 14 comprises a first or proximal end 38, a second opposing or distal end 40, and an inner member main body 42 there between. The first end 38 may comprise a handle or user engaging portion 44. The second end 40 comprises an anatomical engaging component 46 configured to have at least a portion engage with and/or secure to an anatomical structure, such as a vertebral body. The anatomical structure engaging component 46 may comprise an anatomical structure engaging component body 47 which is sized and shaped to fit within the outer member interior region 32 and move within.

The anatomical engaging component 46 may also comprise one or more anatomical structure engaging members, illustrated herein as flanges or cleats 48A and 48B. The flanges or cleats 48A and 48B may be unidirectional. The flanges or cleats 48A and 48B are shown as having a body 50A/50B extending away from the anatomical structure engaging component body 47 and terminating in a terminal point 52A/52B. The terminal points 52A and 52B are sized and shaped to engage with and secure to an anatomical structure. Such shape may be, for example, a pointed end or a rounded end. Preferably, the flanges or cleats 48A and 48B are made from a flexible material so that when the inner member 14 is inserted into and moved within the interior region 32, the flanges or cleats 48A and 48B are oriented or pushed inward (and possibly upward) as a result of a force (contact with the interior region inner surface 54, see FIG. 9), towards the anatomical structure engaging component body 47 or the inner member main body 42, see FIG. 6. When the force is removed, the flanges or cleats 48A and 48B extend outward (or downwardly) away from the anatomical structure engaging component body 47, or the inner member main body 42, see arrows 56, FIG. 7A. While the anatomical structure engaging members are illustrated as flanges or cleats, other structures that engage with and secure to an anatomical structure, this acting as an internal anchor, may be used.

FIG. 7B illustrates an embodiment of the anatomical engaging member 46 having four flanges or cleats, 48A, 48B, 48C and 48D.

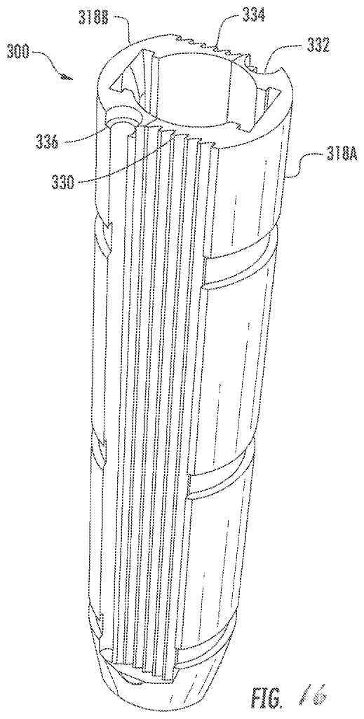

Preferably, the fixation device/shim 10 is used in a bottom-up manner. That is, a user, such as a surgeon, places the surgical retractor fixation device/shim 10 to a desired spot. The surgical retractor fixation device/shim 10 may include the shim inner member 14 already inserted into the shim outer member 12, see FIG. 10A. In this initial position, the surgical retractor fixation device/shim inner member anatomical engaging component 46 is placed in contact with, or near, the bottom surface 58 of the second end 18 of the fixation device/shim outer member 12. The flanges or cleats 48A and 48B are pushed up against or towards the anatomical structure engaging component body 47 and the inner member main body 42 by the interior region inner surface 54. As the user pulls upwards on the surgical retractor fixation device/shim inner member 14, via grabbing or making contact with the handle or user engaging portion 44, see arrows, the flanges or cleats 48A and 48B maintain the closed or non-engaging position, see FIG. 10B. As the user maintains the upward force, see arrows 62, FIG. 10C, the surgical retractor fixation device/shim inner member 14 moves towards the proximal or first end 16 of the surgical retractor fixation device/shim outer member 12. As the flanges or cleats 48A and 48B approach or are positioned within the outer member main body surface openings 36A and 36B, they extend outwardly, forming the second or deployed anatomical structure engaging position. It is in this position that the flanges or cleats 48A and 48B contact or secure to at least a portion of the anatomical structure, thus providing an internal anchor. The flanges or cleats 48A and 48B can be designed to fold back inwards or in line with the surgical retractor fixation device/shim, thus unlocking the flanges or cleats, allowing the surgical retractor fixation device/shim to be removed, which detaches the retractor from the lateral aspect of the vertebral body.

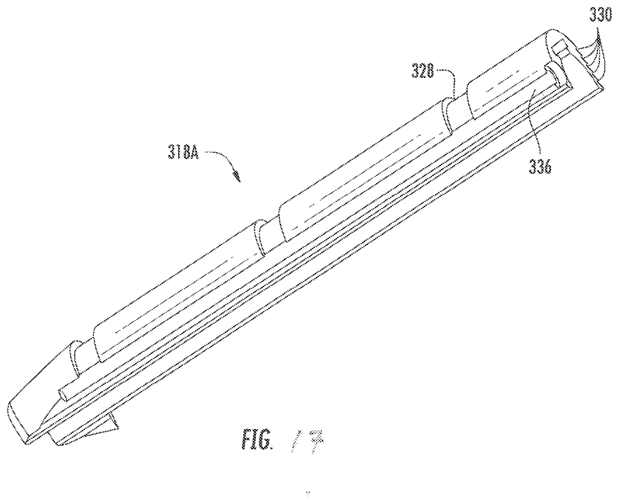

FIG. 11A illustrates an embodiment of the surgical retractor fixation device/shim 10 having inner member guides 64 and 66. The surgical retractor fixation device/shim 10 illustrated in FIG. 11A comprises the same elements or components described above, with the addition of the internal member guides 64 and 66. The internal member guides 64 and 66 are sized and shaped to engage with the flanges or cleats 48A and 48B and guide them towards the outer member main body surface openings 36A and 36B as they are pushed upwardly, see arrows 68. The internal member guides 64 and 66, which are preferably inwardly and angled structures designed to also act as stops by engaging with a portion of the anatomical structure engaging component body 47, thus preventing further upward movement, see FIG. 11B. The internal member guides 64 and 66 may be integrally formed or separate elements.

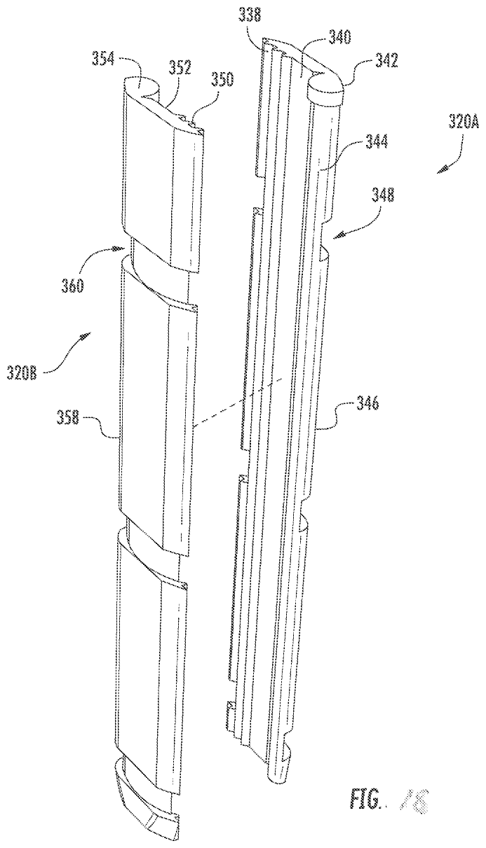

FIGS. 12A-12C illustrate the surgical retractor fixation device/shim 10 used in a top down method, in which the user inserts the surgical retractor fixation device/shim inner member 14 into the surgical retractor fixation device/shim outer member 12 from the first end 16. FIG. 12A illustrates the insertion or first position, with the user pushing the surgical retractor fixation device/shim inner member 14 towards the second or distal end 18, see arrows 70. FIG. 12B illustrates the surgical retractor fixation device/shim 10 used in a top down method, shown moving in a further downward movement, see arrows 72, in a second position prior to deployment of the flanges or cleats 48A and 48B. FIG. 12C illustrates the surgical retractor fixation device/shim 10 used in a top down method, moving in a further downward movement, see arrows 74, shown in a third position, deployment of the inner member flanges or cleats 48A and 48B. The user may move the surgical retractor fixation device/shim inner member 14 upwardly, see arrows 76, to lock the flanges or cleats 48A and 48B in place within the outer member main body surface openings 36A and 36B (after the inner member flanges or cleats 48A and 48B deploy outwardly, see arrows 80 and 82). While the surgical retractor fixation device/shim outer member 12 is shown with internal member guides 64 and 66, the top down deployment approach may be used without them.

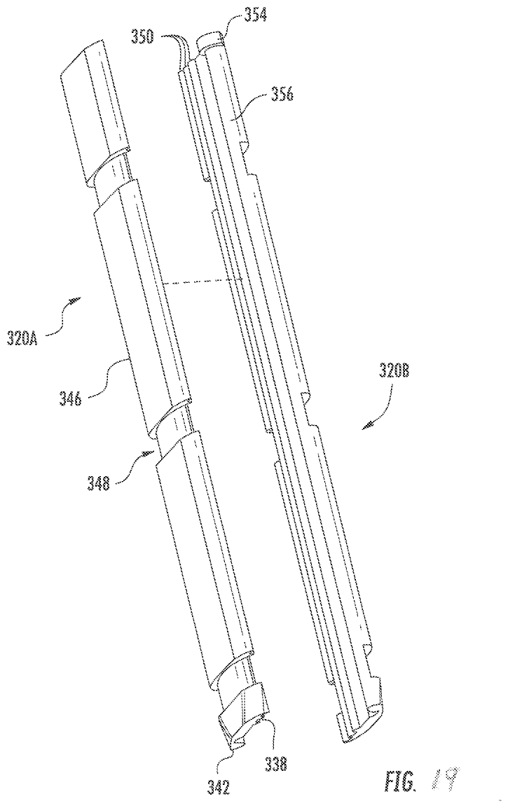

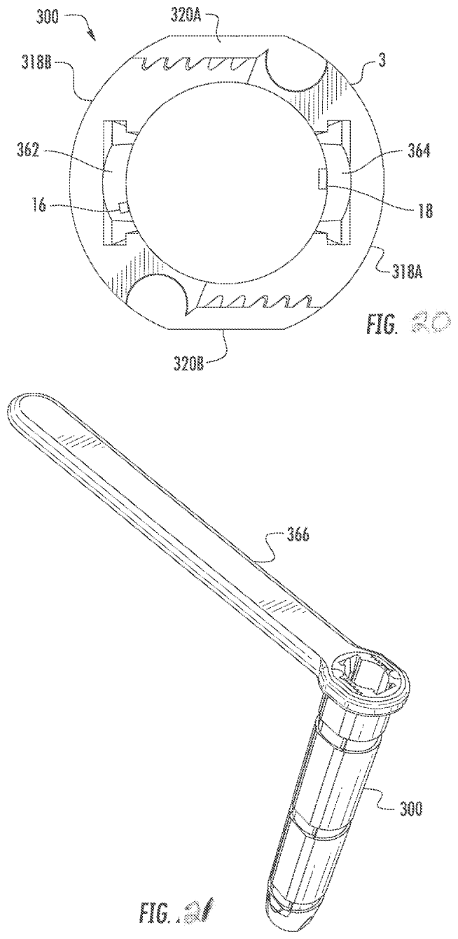

FIGS. 13 and 14 are schematic diagrams illustrating the surgical retractor fixation device/shim 10 shown attached to the spine in a front view (patient positioned on side facing viewer). Referring to FIG. 13, the surgical retractor fixation device/shim 10 is shown inserted within a surgical retractor 84. The second or distal end 18 of the fixation device/shim outer member 12 is shown inserted into a vertebral disc 86. The surgical retractor fixation device/shim inner member 14 is shown in the non-deployed position. FIG. 14 illustrates the surgical retractor fixation device/shim inner member 14 in a deployed position. In this position, the flanges or cleats 48A and 48B are inside of and extend out of the outer member main body surface openings 36A and 36E. The flanges or cleats 48A and 48B are of sufficient size to contact and extend into the vertebral bodies 88 and 90.

The surgical retractor fixation device/shim 10 is designed to engage with any surgical retractor, particularly retractors used for spinal surgical procedures. While the surgical retractor fixation device/shim 10 is described as working with a retractor, the surgical retractor fixation device/shim 10 may engage or secure to the anatomical structure, i.e. the disc, independently. FIG. 15 illustrates an embodiment of the adjustable retractor 300. The retractor 300 illustrated is an elongated body comprising a plurality of engaging segments. The segments may include, for example, a first pair of elongated members 318A and 318B having opposed semi elliptical shaped cross-section and a second pair of elongated members 320A and 320B having opposed semi elliptical or semi cylindrical shaped cross section. Each of the segments has a top, or proximate, portion (forming the proximate end portion 322 of the retractor 300, and a bottom, or distal, portion (forming the distal end portion 324 of the retractor 300). In a particular embodiment, the elongated members 318A and 318B are arranged oppositely about a first plane 321 formed substantially perpendicular to a longitudinal axis 323, and the second pair of elongated members 320A and 320B are arranged oppositely about a second plane 325, the second plane 325 having a different orientation about the longitudinal axis 323 than the first plane 321. That orientation could be, for example, 90 degrees, less than 90 degrees, or greater than 90 degrees.

The length of the elongated members 318A, 318B, 320A, and 320B is preferably of a length to span the distance from adjacent the surgical area at the distal end portion to a location external to the patient. The distal end 324 of the retractor 300, comprised of the distal ends of the elongated members 318A, 318B, 320A, and 320B may be shaped, for example containing a correct concave shape, to allow for more effective dissection of the terminal tissues at the distal end of the retractor 300. When all four elongated members 318A, 318B, 320A, and 320B are configured together, the body 326 of the retractor 300 contains one or more spaced apart recessed channels 328 sized and shaped to receive and hold an elastic member, such as a band. The elongated members 318A, 318B, 320A, and 320B are retained in a fixed position under the influence of elastomeric bands (not shown) that exert a radially directed inward force. Three recessed channels 328 are shown in FIG. 15, by way of example, recognizing that more than three or less than three can be used as well. The elastic bands provide the tension necessary to maintain multiple pieces together during initial insertion of the dilator. They also provide the appropriate amount of tension required to allow sequential dilation/opening of the retractor in parallel fashion along the length of the retractor.

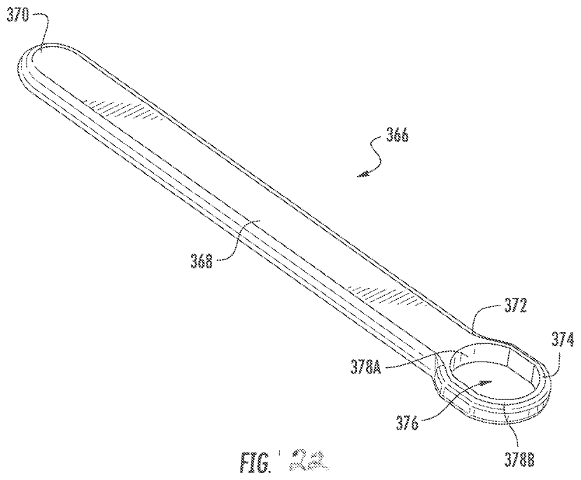

FIG. 16 shows the retractor 300 illustrated in FIG. 15 without the elongated members 320A and 320B. One side of the elongated member 318A contains a plurality of stepped surfaces 330 forming engaging teeth. Opposing the stepped surfaces 330 is a semicircular recessed region 332, see also FIG. 17. The semicircular recessed region 332 preferably begins at or near the proximate portion 322 of the retractor, running the length of the elongated member 318A, and terminating at or near the distal end portion 324. Elongated member 318B contains a similar structure as that described for elongated member 318A. Elongated member 318B contains a plurality of stepped surfaces 334 forming a second set of engaging teeth. Opposing the stepped surfaces 334 is a second semicircular recessed region 336. The semicircular recessed region 336 preferably begins at or near the proximate portion 322 of the retractor, running the length of the elongated member 318B, and terminating at or near the distal end portion 324.

FIG. 18 and FIG. 19 show illustrative embodiments of elongated members 320A and 320B. The elongated member 320A contains a first portion having stepped surfaces 338 positioned within an inner surface 340. The stepped surfaces 338 preferably extend the length of the elongated member 320A. Positioned at the opposite end of the stepped surfaces 338 is a partially cylindrical member 342. The partially cylindrical member 342 protrudes away from the inner surface 340 and has a body 344 which spans the length of the elongated member 320A and is sized and shaped to fit within and engage with the semicircular recessed region 332 of elongated member 318A. Engagement of the partially cylindrical member 342 with the semicircular recessed region 332 of elongated member 318A forms a hinged connection.

The stepped surfaces 338 are sized and shaped to engage with the plurality of stepped surfaces 334 of elongated member 318B. Accordingly, the stepped surfaces 338 and the elongated member 318B cooperate in an interdigiting fashion to restrain lateral contracting of the elongated member 318B with respect to the elongated member 320A. However, the plurality of stepped surfaces 334 and the stepped surfaces 338 yield sufficiently to allow lateral expansion of the retractor 300. This arrangement allows for the elongated member 320A to: 1) hingedly engage with a portion of one elongate member, and 2) expandably engage, via interdigiting cooperation, with a second, independent elongated member. The outer surface 346 contains a plurality of recessed regions 348 which, when assembled, form part of the channel 328 of the retractor 300.

Elongated member 320B contains a first portion having stepped surfaces 350 extending from an inner surface 352. The stepped surfaces 350 preferably extend the length of the elongated member 320B. Positioned at the opposite end of the stepped surfaces 350 is a partially cylindrical member 354. The partially cylindrical member 354 protrudes away from the inner surface 352 and has a body 356 which spans the length of the elongated member 320B. The partially cylindrical member 354 is sized and shaped to fit within and engage with the semicircular recessed region 336 of elongated member 318B. Engagement of the partially cylindrical member 354 with the semicircular recessed region 336 of the elongated member 318B forms a hinged connection.

The stepped surfaces 350 are sized and shaped to engage with the plurality of stepped surfaces 330 of the elongated member 318A. Accordingly, the stepped surfaces 330 and the elongated member 318A cooperate in an interdigiting fashion to restrain lateral contracting of the elongated member 318A with respect to the elongated member 320B. However, the plurality of stepped surfaces 330 and the stepped surfaces 350 yield sufficiently to allow lateral expansion of the retractor 300. This arrangement allows for the elongated member 320B to: 1) hingedly engage with a portion of one elongate member, and 2) expandably engage, via interdigiting cooperation, with a second, independent elongated member. The outer surface 358 contains a plurality of recessed regions 360 which, when assembled, form part of the channel 328 of the retractor 300.

The retractor 300 may be designed to include any feature taught for the various retractors described throughout this document. FIG. 20 illustrates the retractor 300 shown in FIG. 15 having insulated electrical conductors 16 positioned within internal track 362. The insulated electrical conductors 16 may be used, for example, to provide electrical pathways to provide nerve monitoring. An internal track 364 may be used for mounting fiber optical lights 18 to provide illumination of the surgical space located at the distal end of the retractor 300. To aid in any surgical procedure, a hand operated tool 366 may be configured to engage with the retractor 300, see FIG. 21. The tool 366 includes an elongated body 368 having a first end 370 and a second opposing end 372, see FIG. 22. The second end 372 comprises a retractor engaging member 374 having an opening 376. The retractor 300 may be configured to have an exterior surface to operatively engage a complimentary annular surface formed by the hand operated tool 366, whereby the elongate body can be rotated with the patient. As shown in FIG. 22, surfaces 378A and 378B are sized and shaped to engage with elongated members 318A and 318B. Surfaces 380A and 380B are sized and shaped to engage with elongated members 320A and 320B.

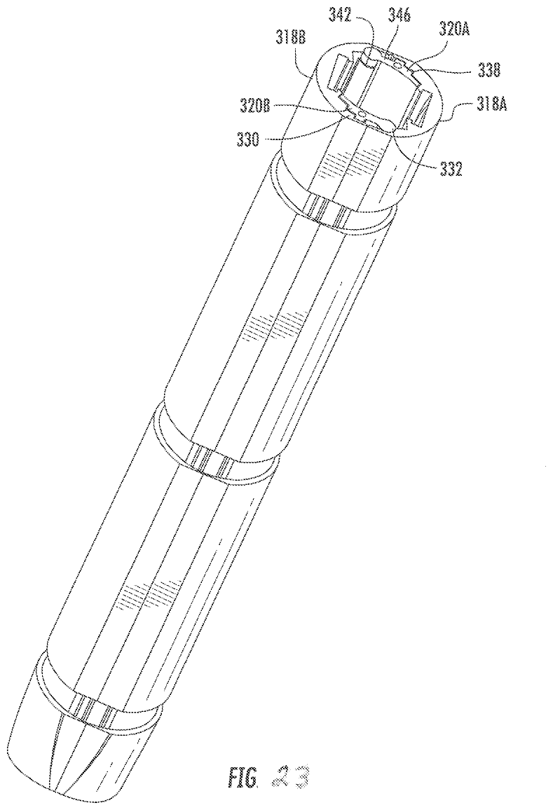

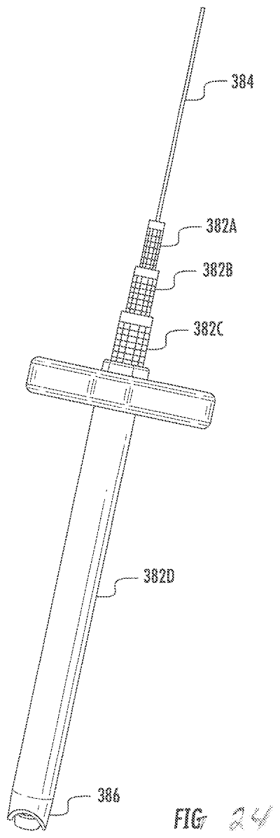

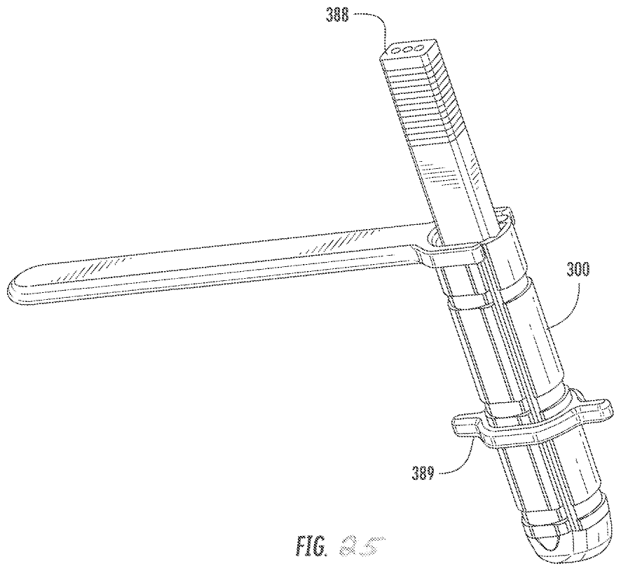

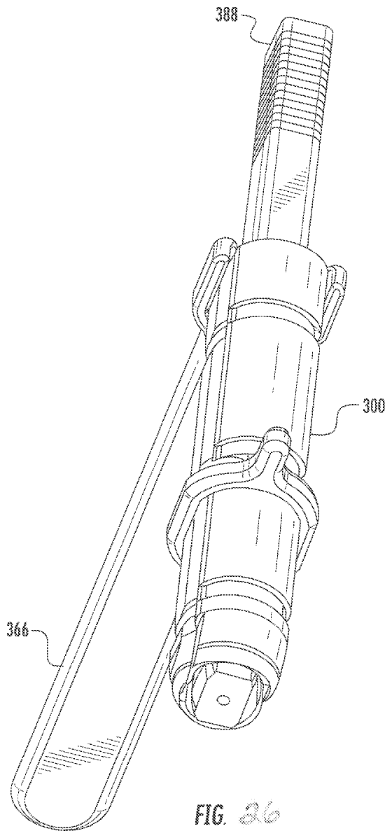

FIG. 23 illustrates the retractor 300 as described above with the elongated members 320A and 320B positioned in the interior portion. In this embodiment, the stepped surfaces 330 of elongated member 318A and the stepped surfaces 334 of elongated member 318A are positioned along the interior surface. In addition, semicircular recessed regions 332 and 336 are also positioned along the interior surface. The elongated member 320A contains the stepped surfaces 338 and the partial cylindrical member 342 positioned within the outer surface 346. The elongated member 320B contains the stepped surfaces 350 and the partial cylindrical member 354 positioned within the outer surface 358.

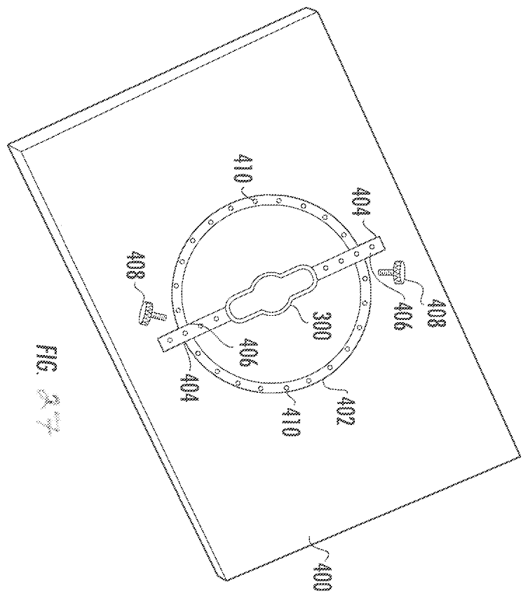

The retractor 300 is designed to be expanded using a plurality of dilators, including the dilators described previously. FIG. 24 illustrates a series of dilators that can be used in the process of inserting the retractor 300. In use, a k-wire 384 inserted into the initial 6 mm dilator 382A can be inserted through a skin incision. A second 9 mm dilator 382B is inserted over the first dilator 382A. A third dilator 282C, 12 mm, is then inserted over the second dilator 382B. All three dilators remain in place until the retractor 300 is slid over and properly placed. A retractor-shaped dilator 382D having a shape similar to, or the same as the retractor 300, may be used to aid in moving nerve fibers. Rotation of the retractor-shaped dilator 382D ninety degrees can function to disassociate final fibers on the spine. The retractor 300 is then placed over the four inner dilators (382A-382D). Once the retractor is rotated and in place, the four inner dilators are removed. A retractor shaped sequential dilator 388 is then inserted into the retractor 300, see FIGS. 25 and 26. The retractor shaped sequential dilator 388 is an elongated body having a shape that mirrors the shape of the inner surface of the retractor 300. For example, the retractor shaped sequential dilator 388 may be shaped or configured to mirror the shape or configuration of one or more of the elongated members 318A, 318B, 320A, and 320B. Such shape allows for expansion of the retractor 300 in one plane or the same direction the retractor 300 is designed to open. Inserting a plurality of differently sized retractor shaped sequential dilators 388, such as a first retractor shaped sequential dilator having a 14 mm height×16 mm length, followed by 14 mm×18 mm, 14 mm×20 mm, and 14 mm×24 mm ensures that the retractor 300 opens in the single intended plane of opening. Retractor 300 may optionally include a key 389 (similar to tool 30) configured to couple to the outer surface. The key 389 is designed to provide a user an additional mechanism to rotate or reposition the retractor 300.

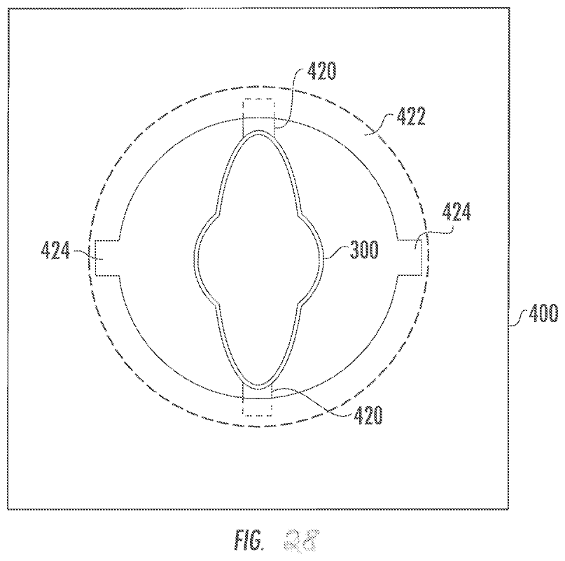

FIG. 27 is a perspective top view of the retractor 300, the patient pad 400, and a device to anchor the retractor to the patient pad. The patient pad 400 eliminates the necessity for a rigid fixation to a point outside of the field of operation or to an independent immobile point, such as a bed rail. The patient pad 400 is applied to the sterilized area on the patient's body. The physical properties of the material, including its size, thickness and composition, cause significant friction, or adhesion, between the pad and the sterile site on the patient's skin. By way of example, patient pad 400 can be reinforced with the application of tape and or IOBAN e if necessary. By way of example, the pad can be formed from a polyurethane material.

Patient pad 400 includes a hard ring 402, formed from metal or plastic, surrounding the retractor 300 that has been placed through the pad 400. A pair of tabs 404 extends from the proximal end of the retractor 300. The material for the tabs 404 is a more malleable composite of the material used for the retractor 300. The tabs 404 include apertures 406 that are configured to receive threaded thumb screws 408. Likewise, the ring 402 includes apertures 410 located around the circumference of the ring 402. Thumb screws 408 are passed through the appropriate apertures 406 in the tabs 404 and are secured in the appropriate apertures 410 located on the ring 402. The tabs 404 can be positioned at the desired location along the ring 402. Then, the thumb screws 408 are positioned in the proper apertures and tightened, whereby the tabs 404 will hold the proximal end of the retractor 300 at the desired angle.

FIG. 28 is a perspective view of an alternative arrangement for anchoring the retractor 300 to the patient pad 400. In this embodiment, the retractor 300 has a pair of radially projecting tabs 420 that are oriented in diametrically opposed relationship. Patient pad 400 includes an annular recess or groove 422 formed either within or recessed on the bottom side of the pad 400. Located on the upper surface of the patient pad 400 is a pair of diametrically opposed slots 424 that extend from the upper surface to the groove or recess 422. To anchor the retractor 300, each of the tabs 420 is brought into alignment with a slot 424. After each tab 420 is positioned within groove 422, the retractor 300 is rotated with respect to the pad 400, thereby effectively anchoring the retractor 300.

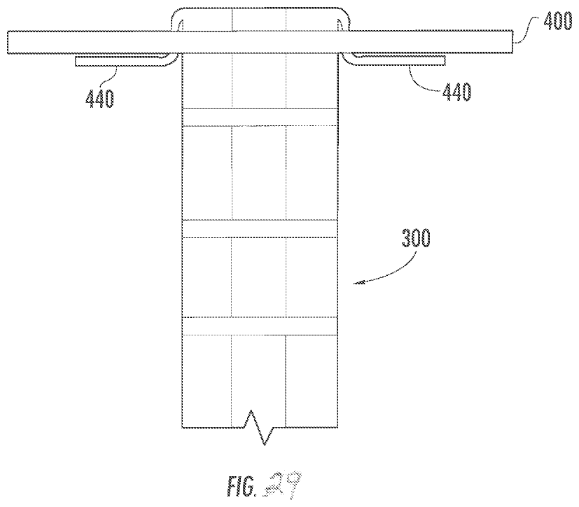

FIG. 29 is a perspective view of another alternative embodiment to anchor the retractor 300 to the patient pad 400. In this embodiment, tabs 440 extend from the proximal aspect of the retractor 300 in diametrically opposed orientation. The tabs 440 are made from a malleable plastic material. As shown in FIG. 27, the tabs 440 fold along the edge of the retractor 300 and the pad 400 is placed over the retractor 300. The retractor 300 extends through the pad 400 through a slit formed therein, while the tabs are positioned beneath the pad 400. The tabs 440 maintain a downward force on the retractor 300 with the constant pressure applied from the patient pad 400 that is in contact with the patient's skin.

FIG. 30 is a perspective view of yet another alternative embodiment to anchor the retractor 300 to the patient pad 400. In this embodiment, the proximal aspect of the retractor 300 includes a series of coarse threads 452 formed on the exterior surface of the retractor 300. The patient pad 400 includes a ring 454 embedded in the upper surface of the patient pad 400. The exterior surface of the ring 454 includes threading 456 configured with the same coarse thread dimension as threads 452. An internally threaded annular ring 450 is used to secure the retractor 300 to the patient pad 400. The internal threads of annular ring 450 are sized and configured to threadably engage threads 452 on the retractor 300 and thread 456 on the embedded ring 454. The retractor 300 is placed through an opening in the patient pad 400 and the annular ring 450 is placed over the retractor 300. As the ring is tightened, it applies a downward force to the retractor's proximal end to maintain the desired depth of the distal tip in the patient's tissue.

Although not illustrated, the surgical retractor fixation device/shim 10 may be configured to attach and/or secure to a surgical table or to the patient pad 400, independently or in combination with the retractor 300. This may be accomplished by direct attachment to the surgical retractor fixation device/shim 10 or through the use of an intermediate structure, such as an extension or arm.

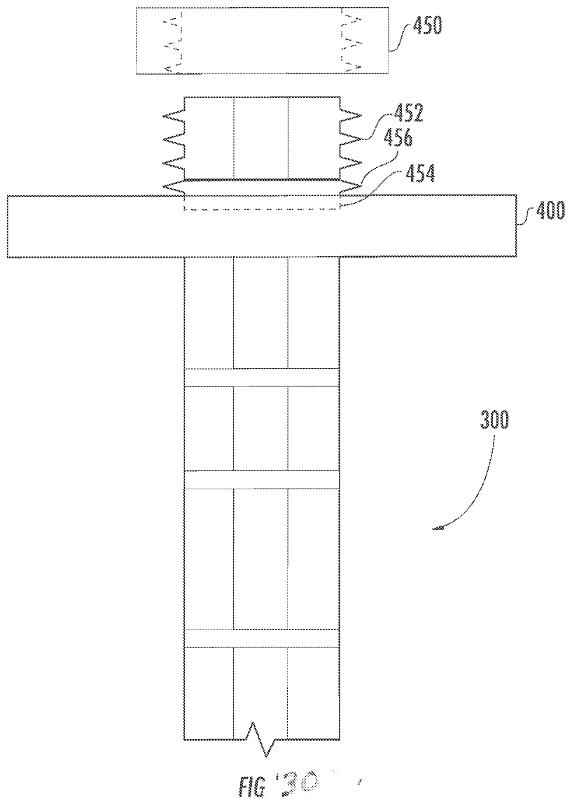

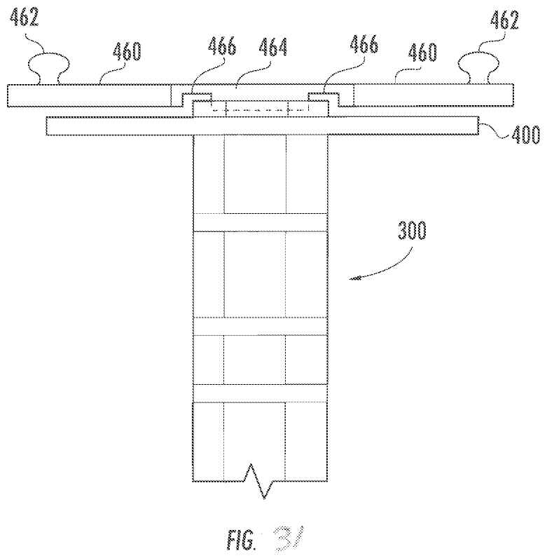

FIG. 31 is a perspective view of a retractor 300 including a gripping element 462, illustrated as a handle knob to manually stabilize the retractor in the patient. In this embodiment, the proximal aspect of the retractor 300 cooperates with a manual anchoring device that includes an annular member 464 with a pair of diametrically opposed arms 460 extending radially outward from annular member 464. The bottom surface of annular member 464 includes an annular groove 466. Annular groove 466 is comprised of a series of semi elliptical segments, configured to mate with the upper surface of the retractor 300, and having sufficient width so as to be able to cooperate with the upper peripheral edge of the retractor 300, either in its compact, intermediate or expanded condition. Arms 460 and annular member 464 are formed from a rigid material, such as a rigid plastic or metal. Each arm 460 includes a gripping element 462. As shown, the gripping element 462 is a ball type handle; however, any other type of handle mechanism, such as a vertical post, or “T” or loop configuration would be acceptable. The retractor 300 would be physically stabilized by an individual grasping and holding either one or both of the gripping elements 462 in a fixed position. Alternatively, one could use the hand stabilized retractor without the aid of the patient pad 400. FIGS. 32-35 illustrate the retractor 300 with surgical retractor fixation device/shim 10. The surgical retractor fixation device/shim 10 is configured to engage and fit or secure within at least a portion of the retractor 300. As illustrated in FIG. 32, the surgical retractor fixation device/shim 10 is designed to engage the retractor 300 through insertion into the internal track 362 or 364. Once inserted, the handle engaging member 22 may rest on the external surface 508 of the retractor 300. As described previously, a portion of the surgical retractor fixation device/shim 10 extends out past the distal end of the retractor 300.

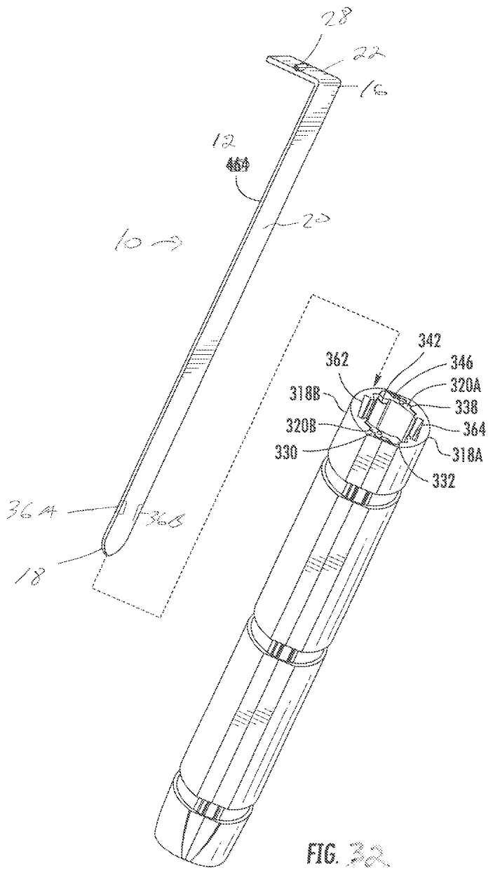

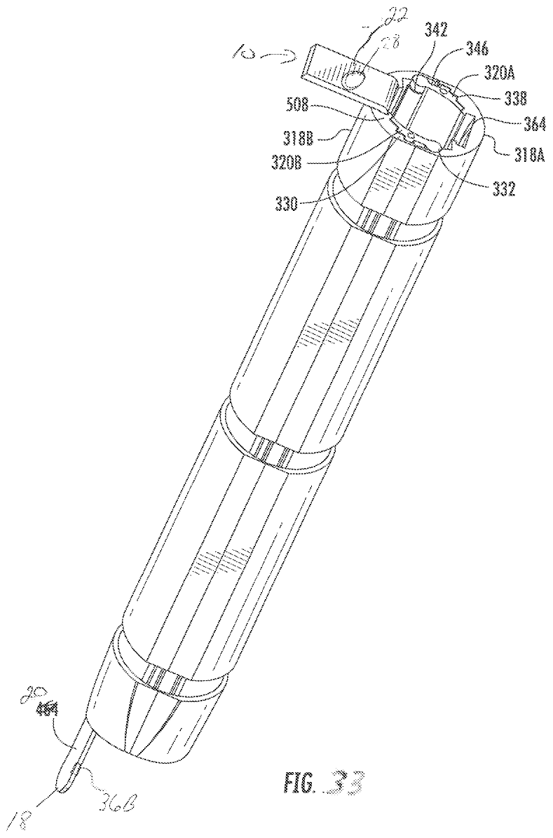

Referring to FIGS. 34-37, an alternative embodiment of the surgical retractor fixation device/shim 10 is shown. The surgical retractor fixation device/shim 10 contains all the same features as previously described with the addition of several components. The surgical retractor fixation device/shim 10 may be cannulated to allow for insertion of one or more devices (referred to also as accessory devices). FIG. 34 illustrates the surgical retractor fixation device/shim outer member 12 having a cannulated passageway 92 (defined by the dashed line) positioned within the outer member main body internal lumen 30, see FIG. 35. The cannulated passageway 92 is defined by the interior region inner surface 54 and a cannula wall 94. The cannulated passageway 92 is sized to preferably run the length of the surgical retractor fixation device/shim 10, but need not. The cannulated passageway 92 comprises a proximal opening 96 and a distal opening 98. The distal opening 98 is shown off center from the distal tip 18. Such positioning is illustrative only, and can be located on center, or anywhere within the outer member main body 20. The cannulated passageway 92 is sized and shaped to allow a device to be inserted, removed, and move within the space. The surgical retractor fixation device/shim shown in FIG. 34 illustrates the outer member main body surface opening 36A (shown in dashed lines) positioned behind the cannulated passageway 92. In FIG. 36 and FIG. 37, the outer member main body surface opening 36A is shown arranged in line with the cannulated passageway 92. In this alignment, the cannula wall 94 may have an opening 100 to allow for the flange or cleat 48A to pass through and into the outer member main body surface opening 36A.

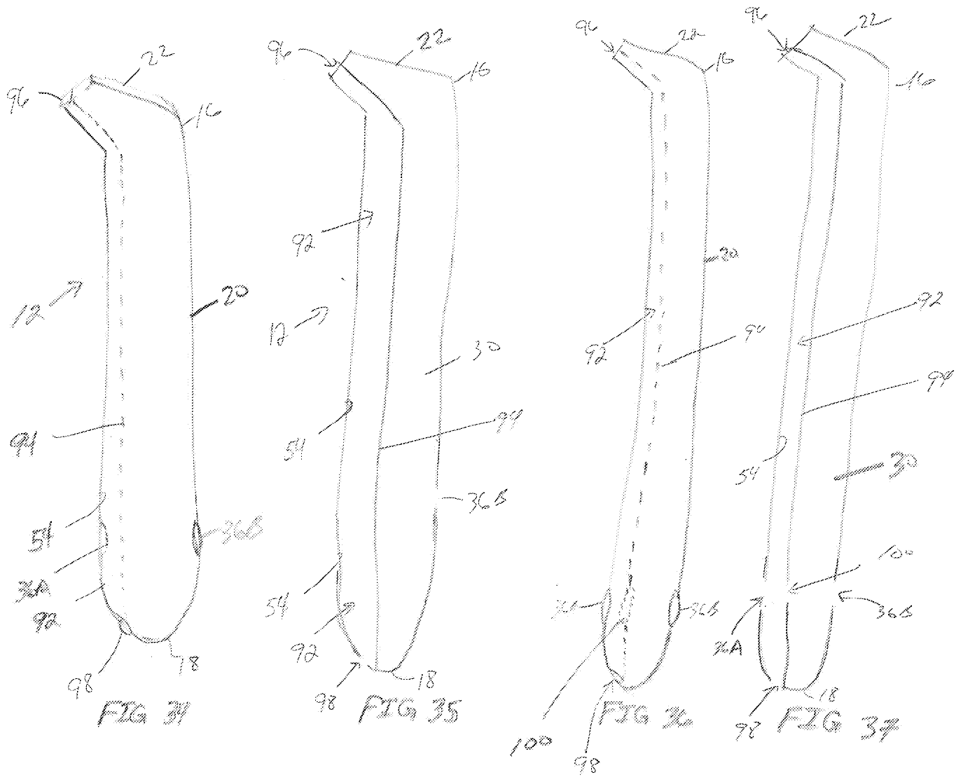

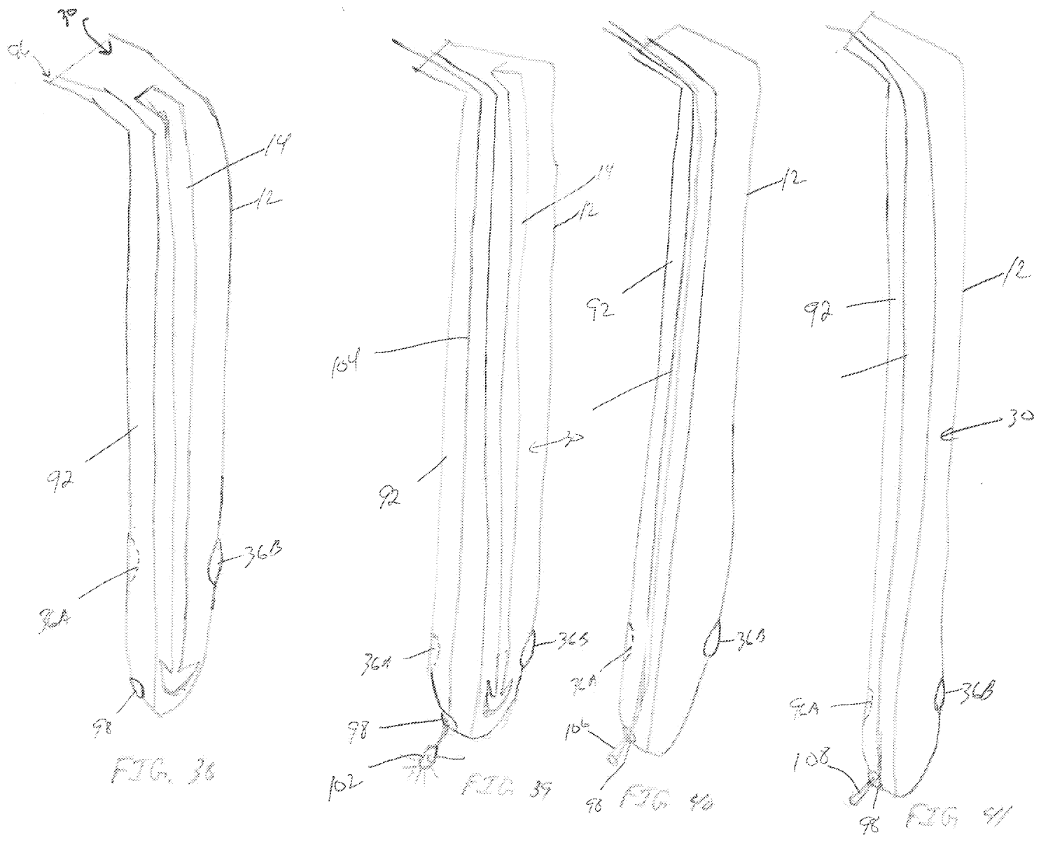

FIG. 38 illustrates the surgical retractor fixation device/shim inner member 14 inserted within the outer member main body internal lumen 30. The surgical retractor fixation device/shim inner member 14 is positioned next to the cannulated passageway 92. FIG. 39 illustrates the insertion of a light source, shown as an LED light 102 with cord 104. The LED light 102 is shown exiting out at the distal opening 98. The LED light 102, however, may be positioned anywhere within the cannulated passageway 92. FIG. 40 illustrates a suction or vacuum line 106 inserted within the cannulated passageway 92. The vacuum line 106 may be attached to a motor or other device configured to provide a vacuum or suction. FIG. 41 illustrates a camera or camera lens 108 inserted within the cannulated passageway 92. FIGS. 39, 40, and 41 are shown without the surgical retractor fixation device/shim inner member 14 inserted therein for illustrative purposes only.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention, and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A surgical retractor fixation shim for use in surgical procedures comprising:
an outer member comprising a first end, a second opposing end, an outer member main body therebetween, and an internal lumen, said outer member main body having an outer surface with one or more surface openings;
an inner member configured to engage with or and fit within said surgical retractor fixation shim outer member comprising a first end, a second opposing end, and an inner member main body therebetween, said second end comprising an anatomical engaging component configured to have at least a portion engage with or secure to an anatomical structure;
wherein, said surgical retractor fixation shim is configured to secure to a portion of said anatomical structure and act as an internal anchor.

2. The surgical retractor fixation shim for use in surgical procedures according to claim 1, wherein said anatomical structure engaging component comprises one or more anatomical structure engaging members.

3. The surgical retractor fixation shim for use in surgical procedures according to claim 2, wherein said one or more anatomical structure engaging members are flanges or cleats.

4. The surgical retractor fixation shim for use in surgical procedures according to claim 3, wherein said flanges or cleats are unidirectional.

5. The surgical retractor fixation shim for use in surgical procedures according to claim 2, wherein said one or more anatomical structure engaging members are made of a flexible material, moving inwardly or outwardly when a force is applied or removed.

6. The surgical retractor fixation shim for use in surgical procedures according to claim 2, wherein said one or more anatomical structure engaging members is sized and shaped to fit within and extend out from said outer member main body one or more surface openings.

7. The surgical retractor fixation shim for use in surgical procedures according to claim 2, wherein said inner member comprises two anatomical structure engaging members.

8. The surgical retractor fixation shim for use in surgical procedures according to claim 2, wherein said inner member comprises four anatomical structure engaging members.

9. The surgical retractor fixation shim for use in surgical procedures according to claim X, wherein said outer member comprises a first outer member main body surface opening sized and shaped to allow a portion of said surgical retractor fixation shim inner member to extend through and rest within and a second outer member main body surface opening sized and shaped to allow a portion of said surgical retractor fixation shim inner member to extend through and rest within.

10. The surgical retractor fixation shim for use in surgical procedures according to claim 1, wherein said outer member inner lumen is hollow or partially hollow to define an interior region.

11. The surgical retractor fixation shim for use in surgical procedures according to claim 1, wherein said outer member first end comprises a handle portion sized and shaped to interact with a portion of a retractor.

12. The surgical retractor fixation shim for use in surgical procedures according to claim 1, wherein said outer member second end comprises a tapered portion configured to engage with a portion of human body.

13. The surgical retractor fixation shim for use in surgical procedures according to claim 1, wherein said outer member main body a length that is sufficient to extend the length of, and out past the distal end of a retractor.

14. The surgical retractor fixation shim for use in surgical procedures according to claim 1; wherein said outer member comprises a cannulated passageway positioned within said outer member main body internal lumen.

15. The surgical retractor fixation shim for use in surgical procedures according to claim 14, wherein said cannulated passageway comprises a proximal opening and a distal opening.

16. The surgical retractor fixation shim for use in surgical procedures according to claim 14, wherein said cannulated passageway comprises a cannula wall.

17. The surgical retractor fixation shim for use in surgical procedures according to claim 16, wherein said cannula wall has an opening sized and shaped to allow for said anatomical structure engaging members to pass through and into said outer member main body surface openings.

18. The surgical retractor fixation shim for use in surgical procedures according to claim 14, wherein said outer member includes one or more accessory devices.

19. The surgical retractor fixation shim for use in surgical procedures according to claim 18, wherein said one or more accessory devices is a camera or camera lens.

20. The surgical retractor fixation shim for use in surgical procedures according to claim 18, wherein said one or more accessory devices is a light.

21. The surgical retractor fixation shim for use in surgical procedures according to claim 18, wherein said one or more accessory devices is an LED light.

22. The surgical retractor fixation shim for use in surgical procedures according to claim 18, wherein said one or more accessory devices suction or vacuum tube.

23. The surgical retractor fixation shim for use in surgical procedures according to claim 1, wherein said surgical retractor fixation shim acts as a spinal internal anchor by being configured to secure internally to a disc space of a spine
